(19) United States
(12) Patent Application Publication
(10) Pub. No.: US 2018/0179094 A1
(43) Pub. Date: Jun. 28, 2018

(54) Dedekahydrogen Hexoxide (H12O6) Life Structure Water

(71) Applicant: Richard McGhee, Oxon Hill, MD (US)

(72) Inventor: Richard McGhee, Oxon Hill, MD (US)

(21) Appl. No.: 15/389,668

(22) Filed: Dec. 23, 2016

(51) Int. Cl. C02F 9/00; A61K 33/00; C01B 15/00

(52) U.S. Cl. CPC C02F 9/00; C02F 1/004; C01B 15/00; A61K 33/00

(57) ABSTRACT

Life structured water is produced by a proprietary process applied by the Hydrogenesis Machine developed by Clinard Wendell Hinson in 2007. The process directly replicates the natural hydrological cycle and includes a replica of the vadose zone, a riverine environment and the trapped atmosphere above an active watercourse replete with the subtle energies of nature which impact the structure and remediate the contamination of tap water using the living energies of nature to remove the chemicals and pathogens rendering pure water hexamers comprised of nothing but hydrogen and oxygen which is uniquely capable of being inducted into the cells which reject low density aqueous solutions of water and so much as a single ion of an chemical and thereby achieves homeodilution, the restoration of vital cellular water.

The apparatus used to make life structured water is the only one of its kind in existence.

INORGANIC CHEMICALS
Parameter MCL (mg/L) MDL (mg/L) Result (mg/L)
Color (units in PCU) 15.00<1
Conductivity (units in μmho) 114.4
Nitrates 10.00 0.3 nd
Total Dissolved Solids 84.5
Turbidity (units in NTU)<1
pH 7.19

### BACKGROUND

### Field of the Invention

The present invention relates to the purification of water and the apparatus for the same. More particularly the present invention relates to the procedure of purification of water which simulates the terrestrial ecosphere of the earth and uses the natural filtering procedure, thereby reorganizing the structure of water magnetically into a six-molecule structure (H12O6) with a frequency of 67 Hz called the water hexamer. Unlike H20 the resulting molecule, devoid of chemical-content, restored of its free-energy and organized into six-fold symmetry is readily inducted into the cells and reverses chronic body drought as define by Dr. Feredoon Batmanghelidj, in “The Body's Many Cries For Water.”

### Background of the Invention

There are 146 ten-thousand trillions tom of water on the earth, 97% of which is seawater which cannot be drunk 2.1% is atmospheric moisture which cannot be captured and less than one percent exists in the form of fresh water lakes, streams and aquifers. Of the final one percent of the earth's water, a substantial amount has been chemically or biologically contaminated by industrialization and associated development. This contamination has met with cellular rejection resulting water management strategies which have been mistakenly identified as disease and has made drinking water contamination one of the highest ranking environmental risks. Clean and pure drinking water is, consequently, in great demand throughout the world, and has rapidly become one of the earth's most prized resources. The fall-out is that billions of people lack a reliable water supply.

This process is designed to process municipal source water which is ostensibly clean. This process oxygenicly transduces all gases, minerals and organics into non-physical matter—energy; all of which is beneficial to the development of adenison tri phosphate, ATP, the fuel of cellular metabolism. Though the earth has surface water in the form of streams, lakes, and wetlands, as well as bays and oceans it has all become contaminated by industrial pollution, auto emissions, drugs and hormones which is not removed by filtering. Surface water also includes the solid forms of water—snow and ice. The water below the surface of the Earth primarily is ground water, but it also includes soil water.

The over arching intent of this invention/process is to produce biologically compatible six-molecule cluster, hexagonal water by restoring tap water to the character of water at the time of creation and can thereby perform the metabolic function which H2O cannot. This is a vital imperative because what goes for water is not water, but low density aqueous solutions which are rejected by the cells which opt for temporary dehydration over chemical contamination which would cause the death of the cell. Life structured water will absorb to the charges on protein surfaces and rebuild water structures which have been lost from the water scavenging which has broken down water-structures in order to make water for digestion and for urine

Generally, as water contamination becomes increasingly severe due to the industrialization and environmental pollution, it costs a great deal to purify the contaminated water. The greatest price of dealing with contamination is health disruption, the second is the waste of source water during processing by reverse osmosis. A study of “Private Drinking Water in Connecticutt produced by the Department of Public Health concludes that 75% of the source water is wasted! This process cleans and organizes every iota of source water In urban areas, many municipal water systems struggle with the ever increasing demand of for available potable water supplies. Additionally, natural inventories of water available for treatments are scarce to find besides, potable usage require more treatment than ever before. Indeed, many areas of the world currently do not have adequate water treatment systems, water utility systems, or basic access to potable water.

Particularly, it is difficult to drink clear water at a local area where tap water is not supplied. Furthermore, even when the tap water is supplied, there are many limitations in using drinking water in an area where water run short. In such situations, it is pertinent to look for innovative ways for ensuring potable/drinking water.

Water purification, or the process of removing undesirable chemicals, biological contaminants, suspended solids and gases from contaminated water, is used to provide water fit for human consumption, also known as potable water or drinking water. Known methods used to purify water include physical processes (i.e. filtration), sedimentation, and distillation, biological processes (i.e. slow sand filters or biologically active carbon), chemical processes (i.e. flocculation and chlorination) and the use of electromagnetic radiation such as ultraviolet (“UV”) light. Water purification may lower the concentration of particulates, like suspended particles, bacteria, parasites, fungi, algae, viruses, and an assortment of dissolved and particulate matter resulting from the environment.

Visual examination Life-structured water is performed by testing standards developed by the Environmental Protection Agency. The optical test is laser based dispersion analysis which concluded that this process produces water that exceeds EPA standard 180.1, testing out at a turbidity of <1.0 and EPA test method SM2510B for conductivity yielding a reading of 114.4. Thus, there is clearly a need for water treatment systems or devices for purifying natural occurring water in order to make it drinkable.

However, none of the existing water purification system replicates the natural system of removal of impurities as is done in an ecosphere.

### OBJECTIVES OF THE INVENTION

The present invention is, therefore, an effort to provide biologically compatible drinking water by replicating the natural hydrologic cycle by which the earth restored contaminated water.

It is still another object of the present invention to provide an apparatus and procedure for producing hexagonally configured, sterile, oxygen-rich free and active useful water capable of being readily inducted into the cells.

It is another object of the present invention to provide an apparatus and procedure for purifying by removing chemicals thereby erasing the memory (chemical signatures) thereby restoring its free energy.

Still another object of the present invention is to provide an apparatus and procedure for purifying water wherein electrochemical dismantlement of ion density thereby maximizing isotopy (residual neutron density) thereby conferring maximal solvent capacity and ionization potential.

Still another object of the present invention is to provide an apparatus and procedure for purifying water by removing the toxic substances, bacteria and fungi present in water naturally.

A further object of the present invention is to provide an apparatus and procedure for purifying water wherein, the beneficial naturally occurring slightly alkaline pH is imparted due to the stabilization of oxygen and its mutants.

A further object of the present invention is to provide an apparatus and procedure for purifying water wherein, the memory of the source water is erased.

Another object of the present invention is to provide an apparatus and procedure for purifying water wherein, the structure of low density aqueous solutions commonly referred to as water is purged of gases, minerals and organics and organized magnetically into a six-molecule structure (H12O6) dodekahydrogen hexoxide, called the water hexamer with a frequency of 67 Hz and having all the properties as described above.

### SUMMARY OF THE INVENTION

An apparatus of the present invention, for purification and obtaining potable water having structural formula (H12O6), the dodekahydrogen hexoxide, comprises of a catalytic reactor (C-Reactor) (1), submicron filter (2), Parabolic tanks (3), Oxidizer (4), Copper coils (5), Storage tanks (6) and control valves (7), wherein the catalytic reactor or the C-reactor (1) is a long cylindrical shaped container having an inlet and an outlet. Both the inlet and outlet are controlled via control valves (7).

The water to be processed enters the said C-reactor (1) from the top of the reactor through the inlet. It gets dispersed uniformly in the catalytic reactor for efficient percolation with the help of dispersion plate. From there, the water passes through the proprietary media zones 1 and zone 2 for effective deionization. Thereafter, the partially purified water exits the said C-reactor (1) through the outlet, and enters the said sub micron filter (2) where the silt is filtered out. After passing through the said submicron filter the filtered water levitates to the said parabolic tanks (3), which are in the shape of a parabola which forms a vertical spin in the collected purified water which had been sprayed onto grounded silver screens, wherein, the filtered water is oxidized with the help of the said oxidizer (4) The water after being oxidized passes through the said copper coils (5) which opens into the said storage tank (6) from where the potable water is pumped to bottling stations.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is an illustration of the apparatus of the water purification apparatus of the present invention.
Process flow diagram ID# lower right of diagram B16-EX1001

FIG. 2 is an enlarged sectional view of the Catalytic reactor of the water purification apparatus according to the present invention.
C-Reactor Assembly diagram ID# lower right of diagram B16-1003 two images.

The image on left is interior look into the reactor

FIG. 2A is an enlarged front perspective view of the Catalytic reactor of the water purification apparatus according to the present invention:
C-Reactor Assembly diagram ID# lower right of diagram B16-1003 two images

The image on right is the frontal perspective view of the C-Reactor.

FIG. 3 is sectional view of the parabolic tanks of the water purification apparatus of the present invention. ID# Lower right: B16 EX 1002

FIG. 4 is an enlarged perspective view of the parabolic tanks of the water purification apparatus of the present invention. ID# Lower right of diagram B14-2000.

### DETAILED DESCRIPTION

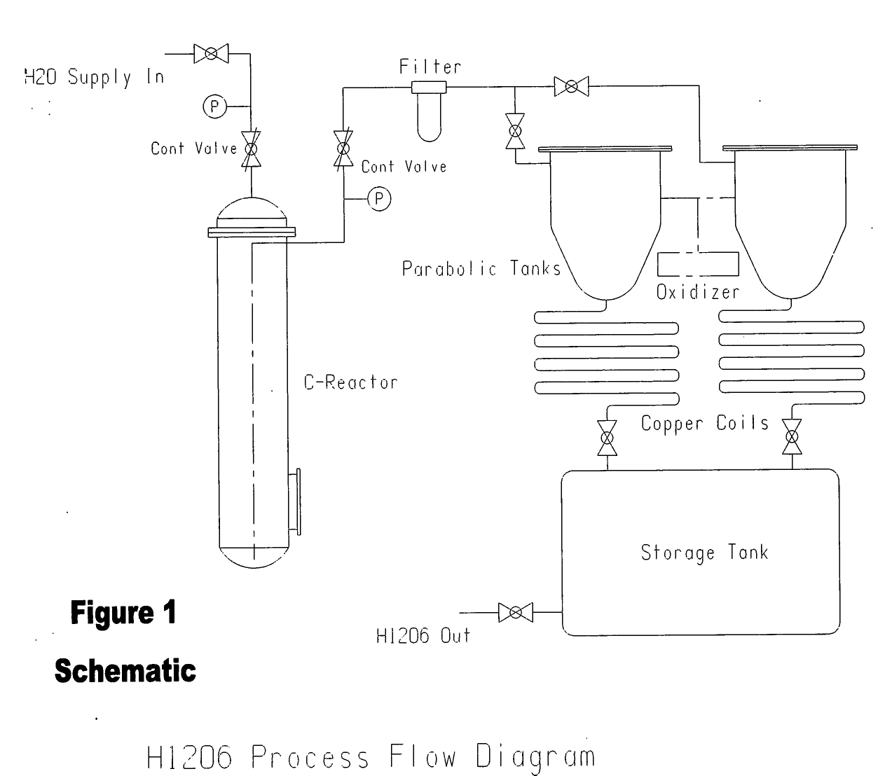

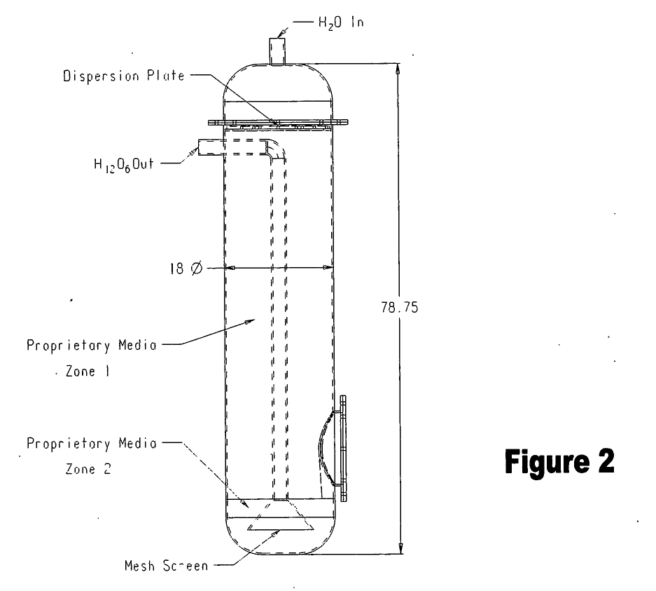

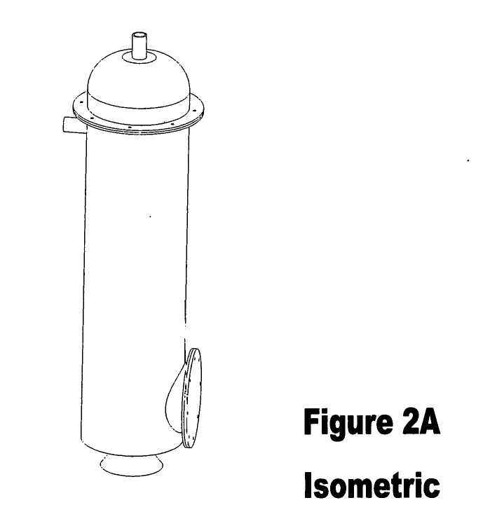

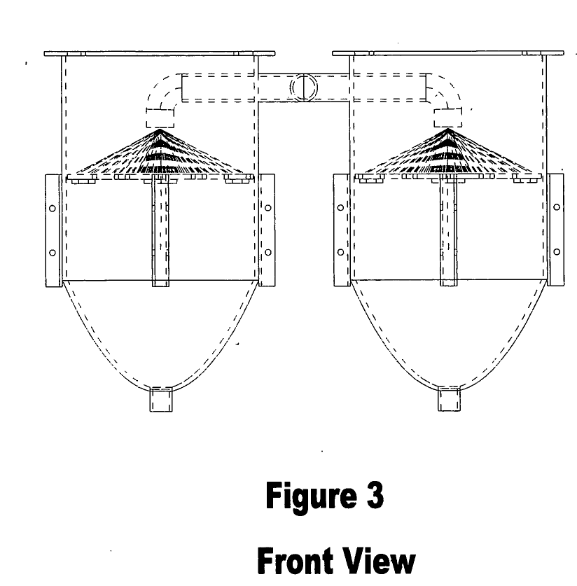

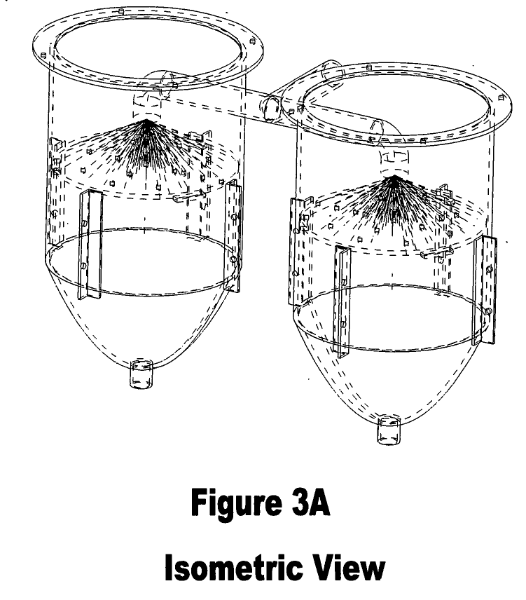

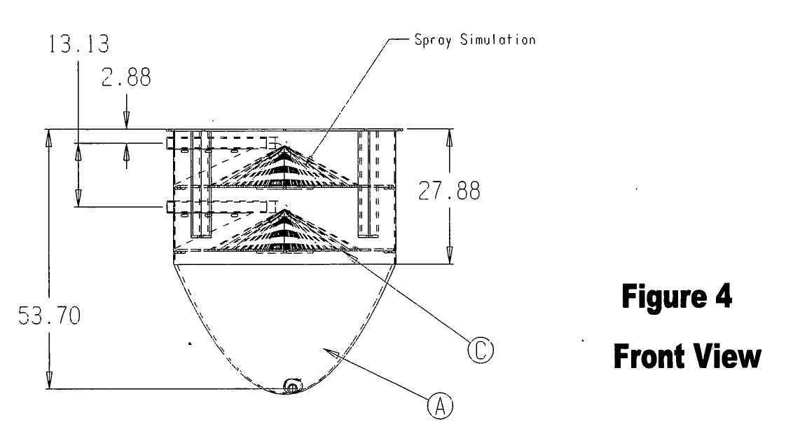

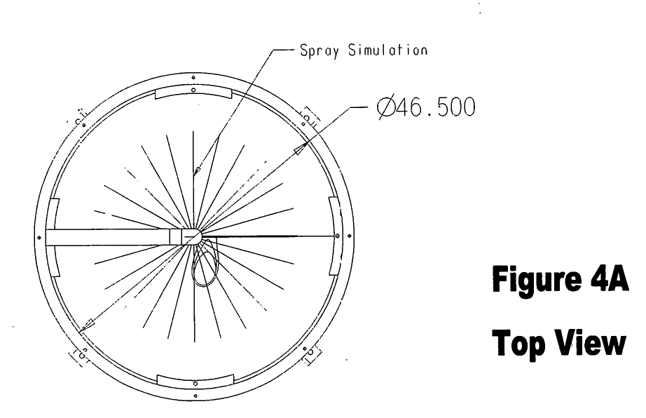

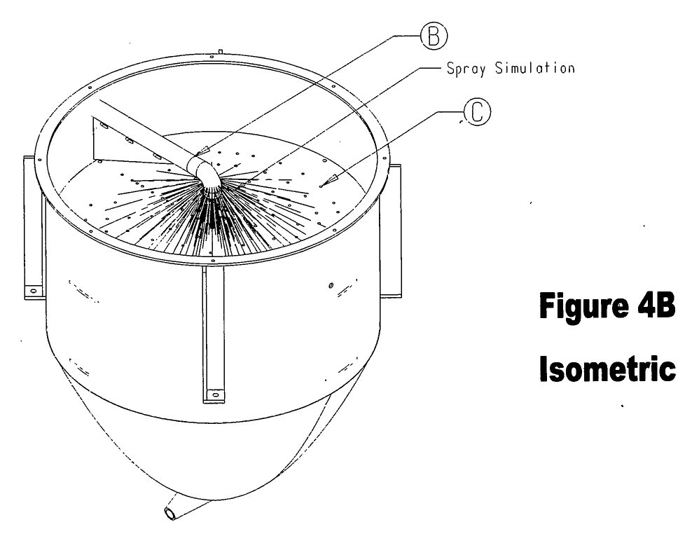

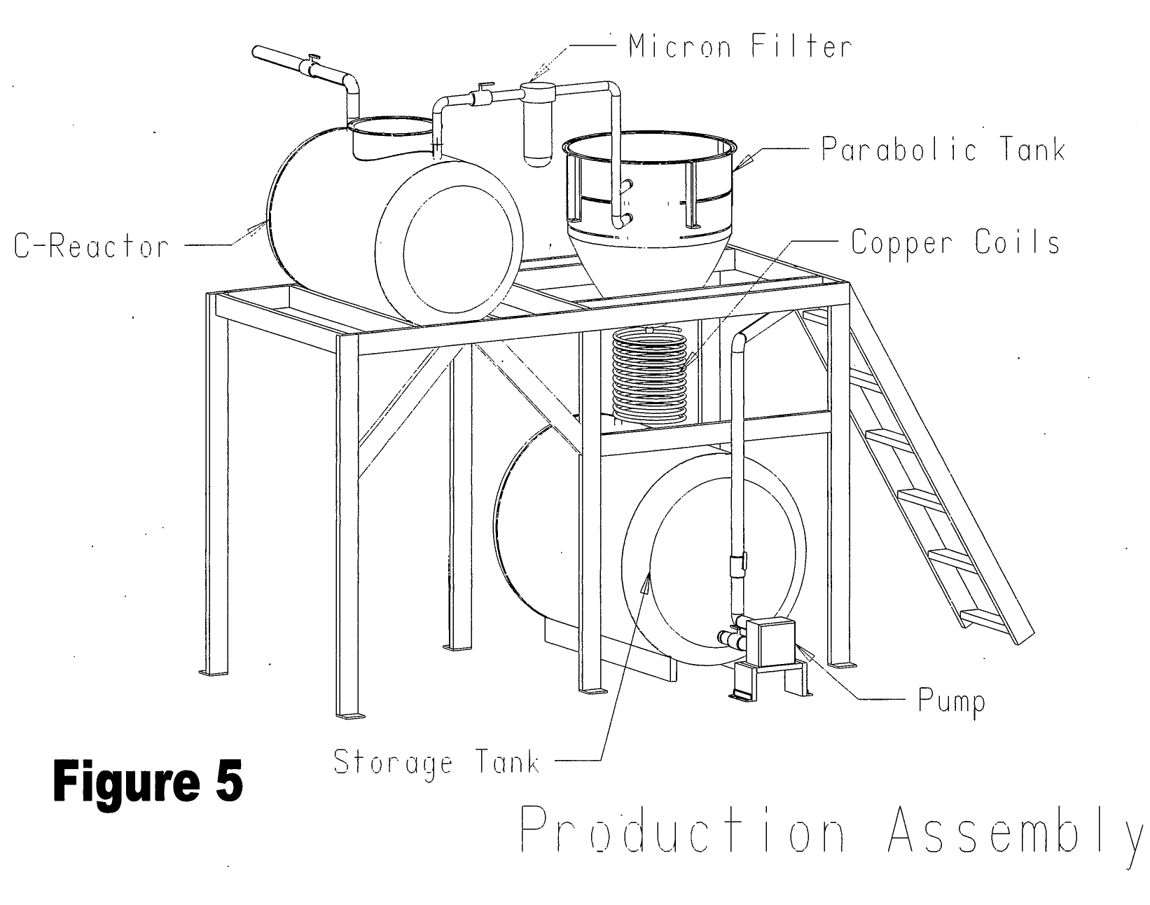

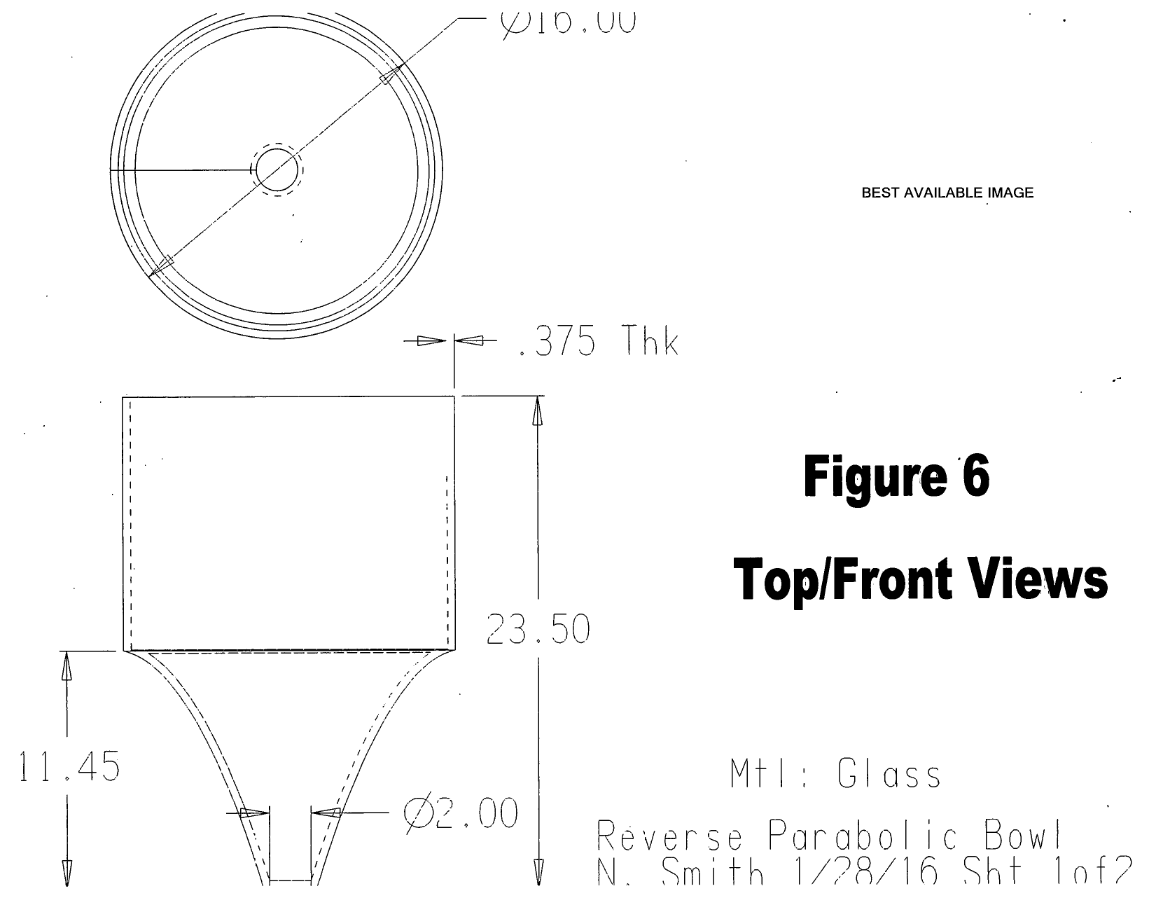

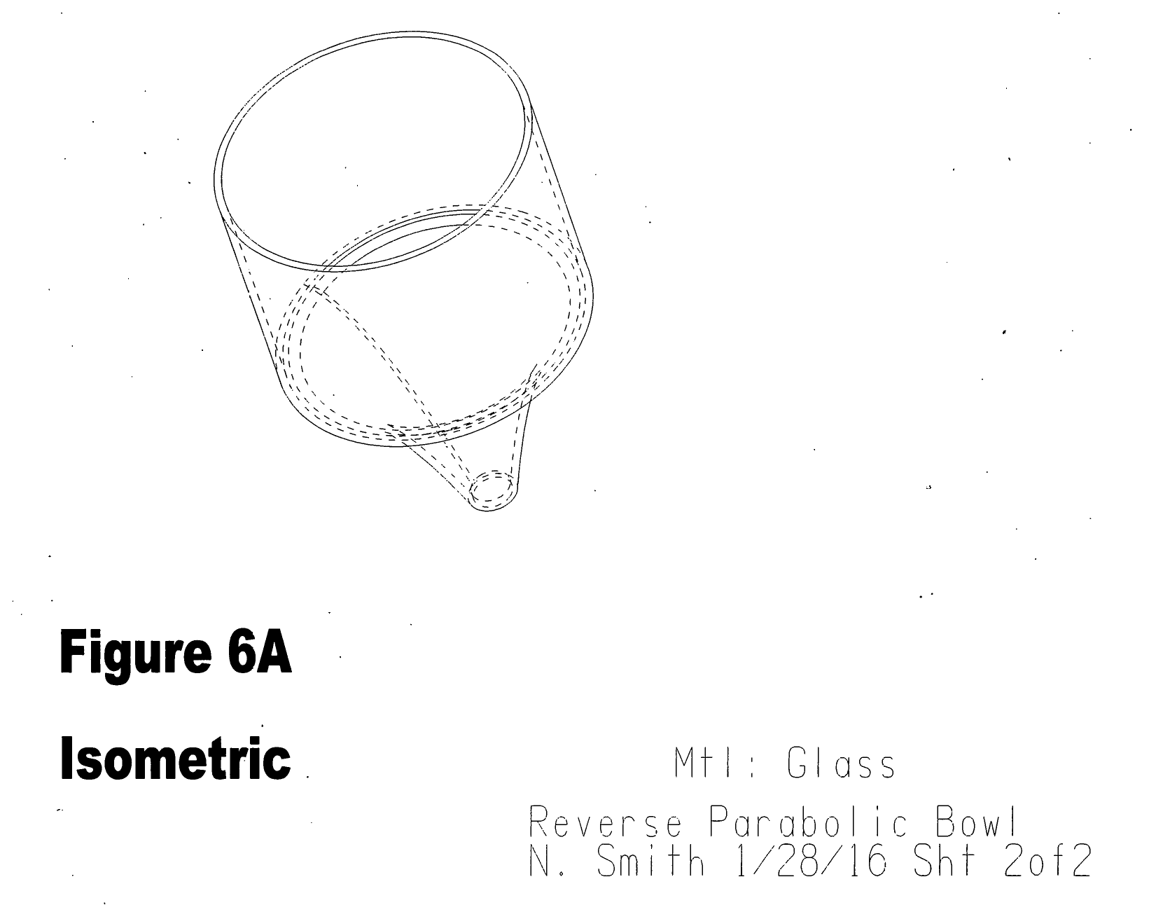

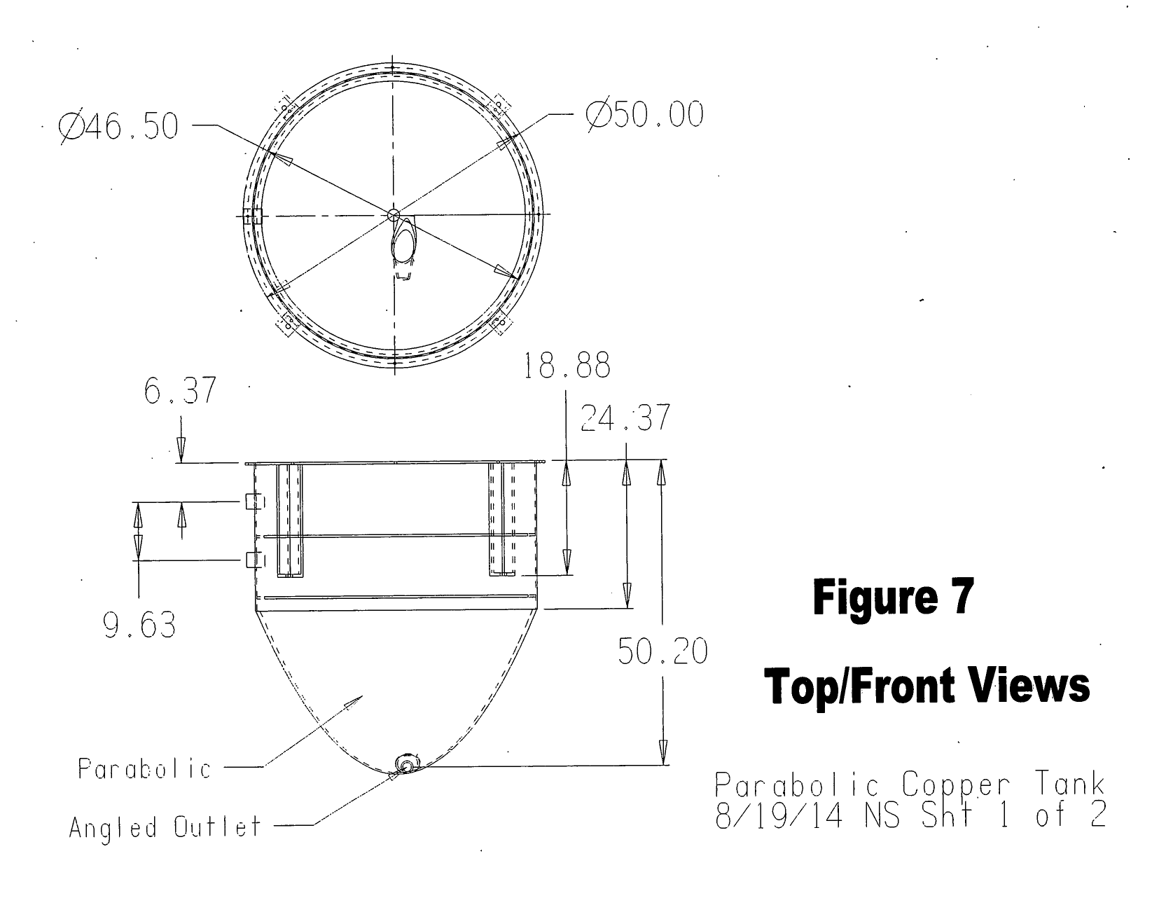

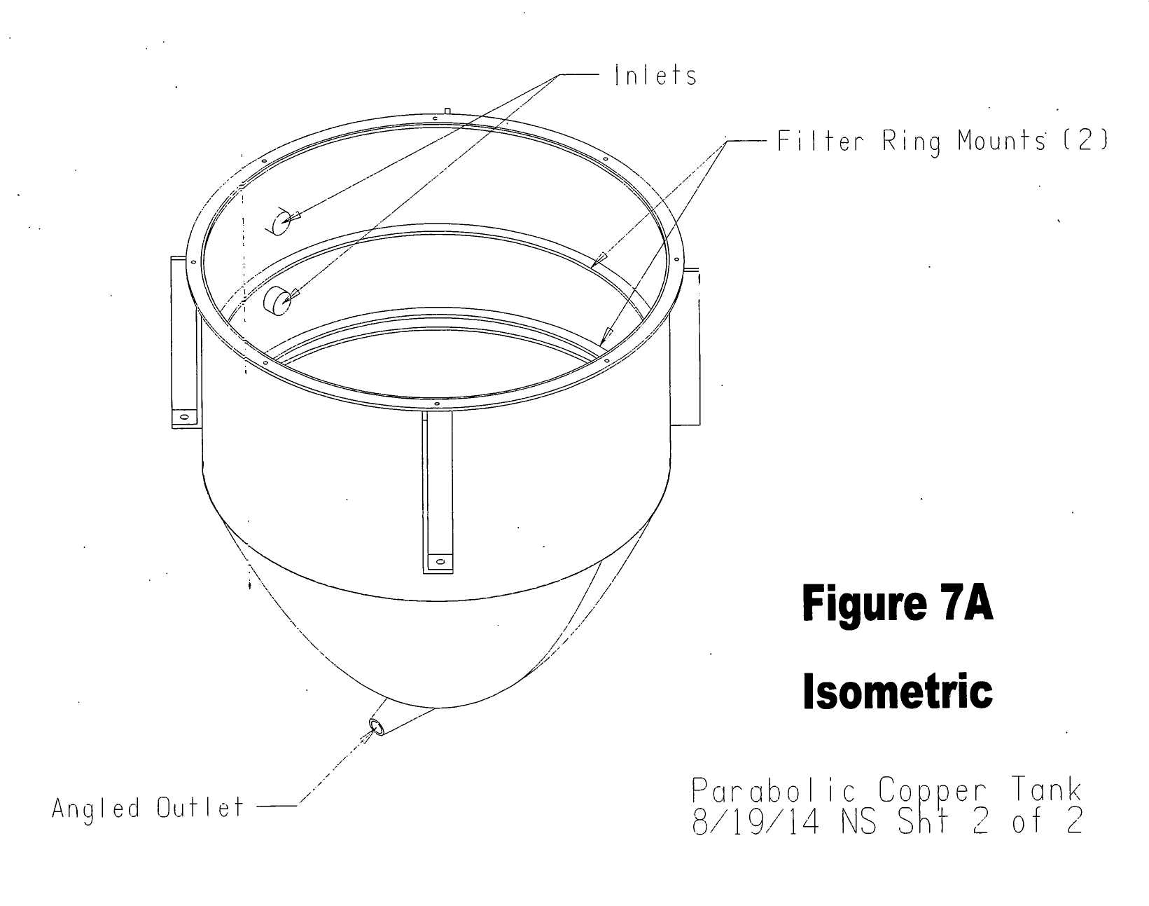

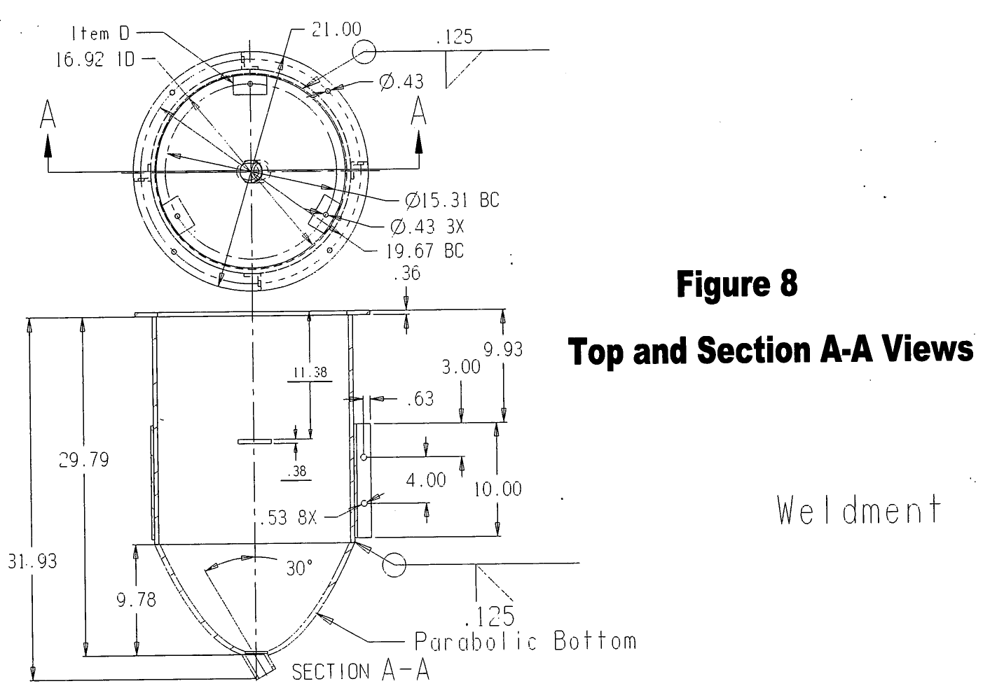

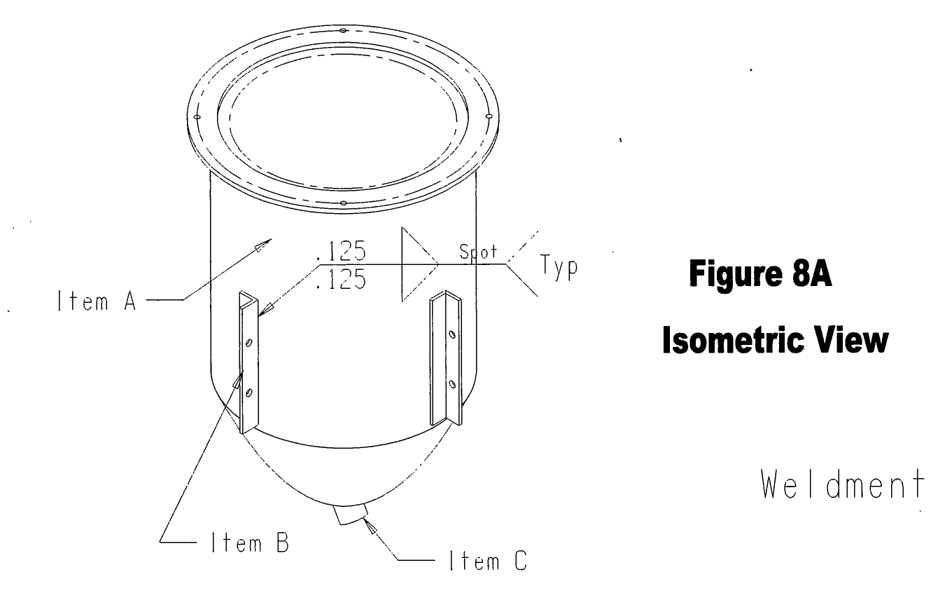

Referring to the drawings, wherein like numerals indicate like elements, that are shown in FIGS. 1-4, an apparatus of the present invention, for purification and obtaining potable water having a six-molecule structure (H12O6) the dodekahydrogen hexoxide, comprising of a catalytic reactor (C-Reactor) (1), submicron filter (2), Parabolic tanks (3), Oxidizer (4), Copper coils (5), Storage tanks (6) and control valves (7).

The catalytic reactor or the C-reactor (1) is a long cylindrical shaped container having an inlet and an outlet. Both the inlet and outlet are regulated via control valves (7). The water to be processed enters the said C-reactor (1) from the top of the reactor through the inlet. It gets dispersed uniformly in the catalytic reactor for efficient percolation with the help of dispersion plate. From there, the water passes through the proprietary media zones 1 and zone 2 for effective deionization. Thereafter the partially purified water the said C-reactor (1) through the outlet, and enters the said sub micron filter (2) where any silt is filtered out. After passing through the said submicron filter the filtered water enters the said parabolic tanks (3), which are in the shape of a parabola, wherein, the filtered water is oxidized with the help of the said oxidizer (4). The water after being oxidized passes through the said copper coils

(5) which opens into the said storage tank (6) from where the potable water is pumped to bottling stations.

The simulation of the natural process of removing water contamination as described in the above process involves various apparatuses and a delicate and intricate process. These are discussed in details as below:

In the first element of the hydrological replication, there is a catalytic tank/reactor which simulates the vadose zone of the earth. In the natural process, precipitated water filters down through the vadose zone to reach the zone of saturation and during this flow the vadose zone filters the surface pollutants.

This above simulation through vadose zone is achieved by the C-Reactor which comprises of:
A dispersion plate which is designed to distribute the water evenly into the circular housing of the catalytic tank for efficient percolation.
For filtration of the water, zeolites and bamboo charcoal are used where they are functioning as proprietary media. These functions are as follows

The Zeolite applied for filtration of water comprises of natural zeolites which are environmentally and economically acceptable hydrated alumina silicate materials with exceptional ion-exchange and sorption properties. These have excellent selectivity for different cations at low temperatures, which is accompanied with a release of non-toxic exchangeable cations (K+, Na+, Ca2+ and Mg2+) to the environment. They are good sorbents of heavy metal cations as well as removes considerable amount of nitrogen from water and adjusts the pH values of water.

Another element utilized in the filtration of water is Bamboo Charcoal which is particularly used due to its porous structure which can absorb and retain impurities easily. Further, bamboo charcoal is rich in a number of minerals including potassium, magnesium, sodium and calcium. As it the water passes through bamboo charcoal apart from filtering water, its minerals are dissolved and it enriches the water with the requisite ion-density which is later transduced into isotopy.

Further, bamboo charcoal has the added benefit of a built-in team of microbes working to decompose toxic substances like tri halo methane and chlorine. These harmless microbes therefore, clear the water naturally of toxic substances, bacteria and fungi.

Bamboo charcoal also releases a type of electromagnetic wave called Far Infrared Waves (FIR), at wavelengths ranging from 4 to 16 micrometres which are absorbed by the human body as well which produce a warming effect on the body, similar to the rays of the sun, and can improve circulation substantially. Upon percolating through the filters zeolite and bamboo charcoal which are herein referred to as proprietary media zones 1 and 2 the partially filtered water enters a pick-up at the bottom of the Catalytic tank, where it levitates past the surface and up into the riverine complex which simulates rainfall, waterfalls, rapids, and whirlpools. Thus by means of an artificial manipulation of the surface area of water, the kinetic energy is channelled into a modification of the water structure. The levitation process raises water from its gravity-dominated state to a metastable equilibrium at a higher energy level.

At this juncture, the highly energized water having the changed structure of (H12O6) exits the riverine complex. The water then enters the parabolic tanks which simulates the terrestrial ecosphere of the earth replete with the simulated atmosphere above a river course under the influence of the short-wave ultra-violet rays of the sun thereby generating the male fertilizing energies of nature which transduce the gases, minerals and organics into non-physical matter which is vitalizing energy.

The terrain of the riverine complex replicates the terrestrial kinetic activities of rainfall, waterfall, whirlpools, rapids under the atmospheric influence of the short wave ultraviolet radiation of the sun, creating the oxygen mutants collectively referred to as the oxygenes which transduce all gases, minerals and organics into non-physical matter; in the form of electrical potential. This electrical potential exists due to the isotopy conferred by the presence of the neutrons of all the chemicals dismantled. The neutrons, having no charge remain in the water as an isotopy.

Once the polar water molecule is divested of chemical contamination, all its free energy is restored as an isotopy, the source of its electrical potential. Therefore, as it spins, it generates magnetic fields which causes it to organize itself into “life structured water” which is six molecule cluster structure. The organizing process erases the memory of formerly held chemical signatures and the absence of gas corrects the specific heat to 97 degrees.

The simulation of the terrestrial ecosphere in the riverine complex is achieved by introducing oxygen into the parabolic vortical chamber where it interfaces with the nanometric sheets of water surface. This causes transduction of ion density into electrical potential and restores the free energy that had previously been bound by the chemical bonds and ion density. As the water folds in on itself to exit the vertical parabolic chamber, the mechanical heat thereby induced, heats up the gases in water with the heat coming from the water causing the water to cool down to the anomaly point of 4 degrees Celsius; at which time all of the oxygen in contact with the surface of the water goes into the water losing both its mass and it's volume, a process referred to as “oxygen stabilization”.

Naturally, the whirlpool of the riverine complex that creates the most efficient means of effective oxygenic transduction through a phenomena referred to as laminar flow. In the present process, the same is re-created through the usage of vortices wherein the water folds in on itself in order to exit the pipe. The water at the outside of the vortex rotates more slowly than at vertical center where the surface of the water in the vertical center folds in on itself as it exits, thereby exposing successive sheets of water-surface to the ionized oxygen resulting in the most effective oxygenic transduction.

As the exiting water folds itself into a spiral, it rotates faster and faster as it moves through the pipe providing an optimal opportunity for water to get transformed into a polar molecule, that organize itself magnetically into six-molecule cluster which is “(Vitale) life structured water” with a frequency of 67 Hz (measured at Duke University by Dr. Anthony Ribiero by NMR Spectroscopy

The magnetic organization of the polar water molecules into free and active useful six-molecule cluster is due to the spinning of these polar molecules.

Once the magnetically oriented, highly oxygenated and energized, six-molecule cluster H12O6 exits the parabolic tanks, it flows out into coiled copper tubes and after exiting from there, enters into the holding/storage tanks which is pumped to bottling stations.

The foregoing descriptions of the present invention have been presented for purposes of illustration and description only. They are not intended to be exhaustive or to limit the present invention to the precise forms disclosed, and obviously modifications remain possible, in particular from the point of view of the design of the various elements or by substitution of equivalent methods, without thus departing from the scope of protection of the invention.

1. The proprietary process for the creation of H12O6) the dodekahydrogen hexoxide molecule.
a catalytic reactor (C-Reactor);
a filter; parabolic tanks; an oxidizer; copper coils;
storage tanks; and
control valves
wherein the said catalytic reactor or the C-reactor is provided with a water inlet and an outlet wherein both the water inlet and outlet are controlled via said cant valves.

2. An apparatus, for purification of water and obtaining potable water having a molecular structure of (H12O6) the dodekahydrogen hexoxide, according to claim 1, wherein the C-reactor is cylindrical in shape.

3. An apparatus, for purification of water and obtaining potable water having a molecular structure of (H12O6) the dodekahydrogen hexoxide, according to claim 1, wherein a dispersion plate is provided within the C-reactor at the top, a short distance away from the water inlet which aids in uniform dispersion and percolation of water.

4. An apparatus, for purification of water and obtaining potable water having a molecular structure of (H12O6) the dodekahydrogen hexoxide, according to claim 1, wherein, one of the process media used in the catalytic reactor is bamboo charcoal.

5. An apparatus, for purification of water and obtaining potable water having a molecular structure of (H12O6) the dodekahydrogen hexoxide, according to claim 1, wherein, one of the process media used in the catalytic reactor is zeolite.

6. An apparatus, for purification of water and obtaining potable water having a molecular structure of (H12O6) the dodekahydrogen hexoxide, according to claim 1, wherein, the said filter is a submicron filter.

7. An apparatus, for purification and obtaining potable water having a molecular structure of (H12O6) the dodekahydrogen hexoxide, according to claim 1, wherein the said parabolic tanks are essentially shaped like a parabola.

8. A process for purification of water and obtaining potable water having molecular structure (H12O6) the dodekahydrogen hexoxide wherein, the water (H2O) enters the C-reactor through the inlet pipe placed at the top of the C-reactor;
wherein, the water gets uniformly dispersed in the catalytic reactor for efficient percolation with the help of dispersion plate;
wherein, the water (H2O) is passed through bamboo charcoal and zeolite where partial filtration of water occurs;
wherein, the above partially filtered water enters the pickup, at the bottom of the catalytic tank, levitating to the top of the catalytic reactor, which energizes the water and wherein this highly energized water having a changed structure of (H12O6) exits the catalytic reactor containing trace amounts of silt;
wherein this high energized water enters the submicron filter where all the solids (silt) are removed;
wherein the water thereby enters the parabolic tanks, which are shaped like a parabola and wherein the terrestrial ecosphere of the earth is simulated and oxygen stabilization of water takes place with the help of the said oxidizer;
wherein, the water, after being oxidized passes through the copper coils which opens into the storage tank from where the potable water is pumped to bottling stations.
